(19) United States
(12) Patent Application Publication
(10) Pub. No.: US 2022/0218595 A1
(43) Pub. Date: Jul. 14, 2022

(54) METHOD OF USING SOOTHING CREAM INCLUDING MACA ROOT EXTRACT AS AN ACTIVE INGREDIENT

(71) Applicant: ANDIVA INC., Chuncheon-si (KR)

(72) Inventors: Young Chul LIM, Chuncheon-si (KR); Jung Ho AHN, Chuncheon-si (KR)

(21) Appl. No.: 17/710,330

(22) Filed: Mar. 31, 2022

Related U.S. Application Data
(62) Division of application No. 16/782,670, filed on Feb. 5, 2020.
Application No. 16/782,670 is a continuation of application No. PCT/KR2019/009490, filed on Jul. 30, 2019.

(30) Foreign Application Priority Data
Aug. 20, 2018 (KR) 10-2018-0096509
Dec. 14, 2018 (KR) 10-2018-0162278

(51) Int. Cl. A61K 8/9789; A61K 8/9794; A61K 8/34; A61K 8/44; A61K 8/49; A61K 8/55; A61K 8/60; A61K 8/65; A61K 8/73; A61K 8/81; A61K 8/891; A61K 8/92; A61K 8/98; A61Q 19/00

(52) U.S. Cl. CPC A61K 8/9789; A61Q 19/007; A61K 8/34; A61K 8/345; A61K 8/44; A61K 8/4926; A61K 8/553; A61K 8/60; A61K 8/65; A61K 8/735; A61K 8/8152; A61K 8/891; A61K 8/922; A61K 8/987; A61K 8/9794

(57) ABSTRACT

Provided is a method of treating a penis of male human using a composition including a Maca root extract as an active ingredient to enhance erection of the penis. The method includes: applying an effective amount of the composition to a skin of a penis of a male human in need thereof. The composition comprises: a skin conditioner, a solvent, a humectant, a viscosity increasing agent, a surfactant, a preservative, a chelating agent, and perfume. The skin conditioner includes: a Maca root extract, a natural extract, niacinamide, glyceryl acrylate/acrylic acid copolymer, hyaluronic acid, arginine, hydrolyzed collagen, sucrose distearate, hydrogenated lecithin, and dimethicone. The composition includes: 0.5 to 10 parts by weight of the Maca root extract; and 1 to 5 parts by weight of the natural extract based on 100 parts by weight of the composition.

### TECHNICAL FIELD

The present invention relates to a soothing cream and a manufacturing method thereof.

### BACKGROUND ART

A soothing cream is also referred to as a moisture cream and is a type of cosmetic products which are applied to the skin to supply moisture to the skin and maintain moisture in the skin. Since dry skin has low flexibility, generates dead skin cells, and may generate skin diseases, etc., problems that may be generated from the dry skin may be overcome by applying an appropriate amount of the soothing cream to the skin.

On the other hand, Maca has been receiving attention as a male health material. Maca, as a brassicaceae plant native to the Andes of South American continent, has been noted for containing abundant protein, unsaturated fatty acid, mineral, etc. Particularly, since it has been known that Maca includes important fatty acids such as linolenic acid, palmitic acid and oleic acid, and an intrinsic unsaturated fatty acid such as macaene or macamide, and Maca is effective in promotion of testosterone secretion, overcoming of menopause obstruction, etc. Therefore, since reproductive ability can be improved when Maca is ingested, or cosmetic products based on Maca are used, a research for developing a composition using Maca has been progressed.

Korean Patent Laid-Open Publication No. 2016-0022172, i.e., a background technique of the present invention, relates to a composition for male health enhancement. The aforementioned Korean Patent Laid-Open Publication No. 2016-0022172 discloses a composition for male health enhancement including a Maca extract and a food or pharmaceutical composition including the composition, but does not disclose a cosmetic product including the Maca extract and effects thereof.

Meanwhile, cosmetic products may be mainly classified into basic cosmetic products for supplying nutrition to the skin and color cosmetic products in which raw materials harmless to the human body are mixed to uniquely express one's outline.

Particularly, the basic cosmetic products include an emulsion, a cosmetic water, a skin, a lotion, etc., and most of women and men use the basic cosmetic products. Such basic cosmetic products perform functions of allowing the skin to be clean by removing dead skin cells and waste of the skin and preventing a skin trouble by forming a thin protective barrier while soothing skin damaged by face washing or the like by adjusting oil and water to be balanced.

Further, investment in cosmetic products for enhancing one's image has recently been increased since the standard of living has been improved as income increases. In addition, interest in and demand for functional cosmetic products related to aging prevention, wrinkle amelioration, whitening, ultraviolet ray blocking and others are on the increase as an aged population increases. However, since compositions for such basic cosmetic products or functional cosmetic products are mostly prepared from chemicals, there are a large number of cases that skin troubles are generated when the compositions are applied to sensitive skins.

On the other hand, active oxygen which is flown in from the outside of hiving body or generated inside the living body becomes the cause of various problems of accelerating aging of the living body and causing cancers. Accordingly, a large number of researches and developments on antioxidants for inhibiting oxidation due to the active oxygen are under way. Although a great deal of effort has conventionally been made to obtain an effect of preventing skin aging by mixing cosmetic materials with antioxidants such as superoxide dismutase (SOD), vitamin E derivatives, ascorbic acid and derivatives thereof, flavonoids, etc., use of the antioxidants is limited since problems are generated from the antioxidants in terms of skin safety or in terms of stability during mixing of the cosmetic materials.

Inflammation occurs for a series of defensive purposes of minimizing reactions of the cells or tissues and recovering damaged areas to the original condition when cells or tissues are damaged by some causes, and inflammation causes functional disorder by inducing humoral and cellular responses to nerves, blood vessels and a lymphatic vessel, thereby causing pain, edema, rubefaction, febrility and others as a result. Factors causing inflammation include physical factors such as external injury, frostbite, burn, radioactivity or the like, chemical factors such as chemicals including acids, and immunological factors such as antibody reactions, and the inflammation occurs even by blood vessels or hormone imbalance in addition to those factors.

Angiectasia occurs by various chemical intermediates secreted from cells damaged by external stimulation, antibody, complement, blood plasma and phagocytic cells crowd into inflammations as permeability increases. Such a phenomenon causes erythema.

Materials performing functions of removing inflammagen and reducing vital reactions and symptoms to eradicate inflammation are referred to as anti-inflammatory agents. Although materials that have been used up to now for the anti-inflammatory purposes include non-steroidal anti-inflammatory agents such as flufenamic acid, ibuprofen, benzydamine, indomethacin, etc., and steroidal anti-inflammatory agents such as prednisolone, dexamethasone, etc., uses of the materials are limited since most of the materials have problems in terms of safety for the skin or in terms of stability when containing cosmetic materials.

In order to solve such problems, a number of researches on cosmetic compositions containing as active ingredients plant extracts of natural materials which have excellent effects in aspects of improving the state of the skin or preventing skin aging or skin damage by having excellent moisturizing, anti-aging, antioxidant and anti-inflammatory effects have recently been progressed. However, there are a large number of cases of showing an insignificant skin improving efficacy when applying the plant extracts alone to the skin.

Therefore, it is required to develop a soothing cream composition containing a natural material extract capable of exhibiting an excellent skin improving effect while improving safety for the skin and stability when containing the cosmetic materials by reducing skin irritation due to chemicals, etc.

### DISCLOSURE

### Technical Problem

The present invention is to solve problems of the aforementioned existing technology, and an object of the present invention is to provide a soothing cream.

Further, the other objective of the present invention is to provide a method of manufacturing the soothing cream.

However, technical problems to be achieved by embodiments of the present invention are not limited to the aforementioned technical problems, and other technical problems may exist.

### Technical Solution

As a technical means for achieving the aforementioned technical objects, a first aspect of the present invention provides a soothing cream including a skin conditioner, a solvent, a humectant, a viscosity increasing agent, a surfactant, a preservative, a chelating agent, and perfume, in which the skin conditioner includes a Maca root extract, a natural extract, niacinamide, glyceryl acrylate/acrylic acid copolymer, hyaluronic acid, arginine, hydrolyzed collagen, sucrose distearate, hydrogenated lecithin, and dimethicone.

According to an embodiment of the present invention, although the soothing cream may include 0.5 to 10 parts by weight of the Maca root extract and 1 to 5 parts by weight of the natural extract based on 100 parts by weight of the soothing cream, the present invention is not limited thereto.

According to an embodiment of the present invention, although the natural extract may include a material selected from the group consisting of a snail mucus filtrate, a Morus alba root extract, a green tea extract, a persimmon leaf extract, an aloe vera leaf extract, a chamomile flower extract, a Portulaca oleracea L. extract, Argania spinosa kernel oil, and combinations thereof, the present invention is not limited thereto.

According to an embodiment of the present invention, although the skin conditioner may additionally include a material selected from the group consisting of butylene glycol, purified water, 1,2-hexanediol, glycerin, and combinations thereof, the present invention is not limited thereto.

According to an embodiment of the present invention, although the Maca root extract may include a material selected from the group consisting of a Maca powder, a Maca hot water extract, and a combination thereof, the present invention is not limited thereto.

According to an embodiment of the present invention, although the solvent may include purified water, glycerin, 1,2-hexanediol and butylene glycol, the present invention is not limited thereto.

According to an embodiment of the present invention, although the humectant may include a material selected from the group consisting of propylene glycol, glycerin, and a combination thereof, the present invention is not limited thereto.

According to an embodiment of the present invention, although the soothing cream may further include a viscosity increasing agent including a material selected from the group consisting of carbomer, hydroxyethyl cellulose, ammonium acryloyldimethyltaurate/VP copolymer and combinations thereof, and a surfactant including PEG-60 hydrogenated castor oil and sucrose distearate, the present invention is not limited thereto.

According to an embodiment of the present invention, although the soothing cream may further include a preservative including chlorphenesin, and a chelating agent including disodium EDTA, the present invention is not limited thereto.

According to an embodiment of the present invention, although the soothing cream may further include menthol as the skin conditioner, the present invention is not limited thereto.

According to an embodiment of the present invention, although the soothing cream may include 0.1 to 2 parts by weight of menthol based on 100 parts by weight of the soothing cream, the present invention is not limited thereto.

Furthermore, a second aspect of the present invention provides a method of manufacturing a soothing cream, the method including the steps of: preparing a first mixture including purified water, propylene glycol, glycerin, a Maca root extract, butylene glycol, 1,2-hexanediol, niacinamide, a snail mucus filtrate, glyceryl acrylate/acrylic acid copolymer, hyaluronic acid, arginine, a Morus alba root extract, a green tea extract, a persimmon leaf extract, an aloe vera leaf extract, a chamomile flower extract, a Portulaca oleracea L. extract, hydrolyzed collagen, a viscosity increasing agent, a chelating agent, and a preservative; mixing a second mixture including ethanol, 1,2-hexanediol, PEG-60 hydrogenated castor oil, perfume and Argania spinosa kernel oil with the first mixture; and mixing a third mixture including hydrogenated lecithin, purified water, glycerin, dimethicone, and sucrose distearate with the first mixture.

According to an embodiment of the present invention, although the step of preparing the first mixture may include the steps of: preparing a first material including the Maca root extract, butylene glycol, purified water and 1,2-hexanediol; preparing a second material including the glycerin, purified water, glyceryl acrylate/acrylic acid copolymer and 1,2-hexanediol; preparing a third material including the purified water, butylene glycol, Morus alba root extract, green tea extract, persimmon leaf extract and aloe vera leaf extract; and mixing the first material, second material, third material, purified water, propylene glycol, glycerin, niacinamide, snail mucus filtrate, hyaluronic acid, arginine, chamomile flower extract, Portulaca oleracea L. extract, hydrolyzed collagen, viscosity increasing agent, chelating agent and preservative, the present invention is not limited thereto.

According to an embodiment of the present invention, although the step of preparing the first mixture may be performed at a temperature of 65 to 80° C., the present invention is not limited thereto.

According to an embodiment of the present invention, although the step of injecting the second mixture may further include a step of injecting menthol, the present invention is not limited thereto.

The aforementioned means for solving problems is only illustrative and should not be interpreted to be intended to limit the present invention. In addition to the aforementioned exemplary embodiments, additional embodiments may exist in drawing and the detailed description of the present invention.

### Advantageous Effects

According to the aforementioned means for solving problems of the present invention, a soothing cream according to the present invention is nontoxic and does not have side effects with the human body since the soothing cream includes a natural extract.

Further, since the natural extract of a soothing cream according to the present invention includes a snail mucus filtrate, a Morus alba root extract, a green tea extract, a persimmon leaf extract, an aloe vera leaf extract, a chamomile flower extract, a Portulaca oleracea L. extract, and Argania spinosa kernel oil, the soothing cream according to the present invention may have excellent effects of moisturizing and soothing the skin.

Further, when a soothing cream according to the present invention is applied to male genitalia, it can be confirmed that length of the male genitalia is extended.

However, effects that can be obtained from the present invention are not limited to the aforementioned effects, and other effects may exist.

### DESCRIPTION OF DRAWINGS

FIG. 1 is a flowchart of a method of manufacturing a soothing cream according to an embodiment of the present invention.

### BEST MODE

Hereinafter, examples of the present invention will be described in detail with reference to the annexed drawing so that those skilled in the art will easily be able to implement the present invention.

However, the present invention may be implemented in various forms and not limited to the examples described herein. A part having no relationship with the description is omitted to clearly describe the present invention in the drawing, and a similar constituent element is indicated by a similar reference numeral throughout the present specification.

In the whole present specification, when a part is referred to as to be “connected” to the other part, the parts are not only “directly connected” to each other, but also “electrically connected” to each other while interposing another part therebetween.

In the whole present specification, when any member is positioned “on”, “over”, “above”, “beneath”, “under”, and “below” the other member, this not only includes a case that the any member is brought into contact with the other member, but also includes a case that another member exists between two members.

In the whole present specification, if a prescribed part “includes” a prescribed element, this means that another element can be further included instead of excluding other elements unless any particularly opposite description exists.

When unique manufacture and material allowable errors of numerical values are suggested to mentioned meanings of terms of degrees used in the present invention such as “about”, “substantially”, etc., the terms of degrees are used as the numerical values or as a meaning near the numerical values, and the terms of degrees are used to prevent that an unscrupulous infringer unfairly uses a disclosure content in which extract or absolute numerical values are mentioned to help understanding of the present invention. Further, in the whole present specification, “a step doing ˜” or “a step of ˜” does not mean “a step for ˜”.

In the whole present specification, a term of “a combination thereof” included in a Markush type expression, which means a mixture or combination of one or more selected from the group consisting of elements described in the Markush type expression, means including one or more selected from the group consisting of the elements.

In the whole present specification, a description of “A and/or B” means “A or B”, or “A and B”.

Hereinafter, a soothing cream according to the present invention and a manufacturing method thereof will be described specifically with reference to embodiments, examples and drawing. However, the present invention is not limited to such embodiments, examples and drawing.

As a technical means for achieving the aforementioned technical objects, a first aspect of the present invention provides a soothing cream including a skin conditioner, a solvent, a humectant, a viscosity increasing agent, a surfactant, a preservative, a chelating agent, and perfume, in which the skin conditioner includes a Maca root extract, a natural extract, niacinamide, glyceryl acrylate/acrylic acid copolymer, hyaluronic acid, arginine, hydrolyzed collagen, sucrose distearate, hydrogenated lecithin, and dimethicone.

Generally, a soothing cream means a cosmetic product for supplying moisture to the skin or maintaining the moisture. For the purposes of supplying and maintaining moisture, the soothing cream may include a skin conditioner including a material for moisturizing the skin, a solvent for dissolving the skin conditioner, a humectant which is injected into the solvent to maintain moisture of the solvent, a viscosity increasing agent which increases viscosity of the soothing cream such that the soothing cream does not flow down from the skin, a surfactant for mixing of the skin conditioner and the solvent, a preservative for inhibiting decomposition of the soothing cream, a chelating agent for preventing very small amounts of metal ions from flowing into the soothing cream, and perfume for emitting incense.

Although a surfactant according to the present invention may include a cleaning agent for removing contaminants in the soothing cream, a solubilizing agent for transparently dissolving a small amount of oil in the solvent, an emulsifier for allowing the oil and solvent to be mixed well, a foam promoter for increasing efficacy of other surfactant by forming bubbles, etc., the present invention is not limited thereto.

A skin conditioner according to the present invention means a material for skin moisturizing and soothing effects and skin health amelioration. The skin conditioner may include a moisturizer or humectant which allows moisture to be remained on the skin by pulling moisture in the air, an emollient or occlusive which suppresses evaporation of moisture by forming an oil film on the skin, miscellaneous substances for giving special ingredients to the skin, etc.

According to an embodiment of the present invention, although the soothing cream may include the Maca root extract, natural extract, niacinamide, glyceryl acrylate/acrylic acid copolymer, hyaluronic acid, arginine, hydrolyzed collagen, sucrose distearate, hydrogenated lecithin, and dimethicone as active ingredients, the present invention is not limited thereto.

According to an embodiment of the present invention, although the Maca root extract may include a material selected from the group consisting of a Maca powder, a Maca hot water extract, and a combination thereof, the present invention is not limited thereto.

A Maca root extract according to the present invention means an ingredient extracted from the root of Maca (Lepidium meyenii Walp), i.e., a brassicaceae plant. The Maca contains important fatty acids such as linolenic acid, palmitic acid and oleic acid, plant sterol, and mineral in large amounts, and may contain an intrinsic unsaturated fatty acid such as macaene or macamide which is not discovered from other plants. Such ingredients have been reported to have biological activities. Particularly, it has been reported that the Maca has a glucosinolate content 100 times higher than those of other brassicaceae plants, and glucosinolate controls the immune system and has an anti-cancer effect.

The Maca also has functions of promoting blood circulation and enhancing erection of the penis of male by stimulating artery positioned in the penis of male.

According to an embodiment of the present invention, although the soothing cream may include 0.5 to 10 parts by weight of the Maca root extract and 1 to 5 parts by weight of the natural extract based on 100 parts by weight of the soothing cream, the present invention is not limited thereto.

According to an embodiment of the present invention, although the natural extract may include a material selected from the group consisting of a snail mucus filtrate, a Morus alba root extract, a green tea extract, a persimmon leaf extract, an aloe vera leaf extract, a chamomile flower extract, a Portulaca oleracea L. extract, Argania spinosa kernel oil, and combinations thereof, the present invention is not limited thereto.

A snail mucus filtrate according to the present invention means a material obtained by purifying mucus secreted by a snail. The snail mucus filtrate is very excellent in a moisturizing effect and a function of protecting the skin from external stimulation and excellent in prevention of aging and maintenance of skin elasticity by mucin ingredient.

A Morus alba root extract according to the present invention is extracted from Morus alba root, brightens tone of the skin, and executes an anti-oxidation activity.

A green tea extract according to the present invention means an ingredient extracted from green tea leaves. The green tea extract has an anti-oxidation activity, an anti-cancer activity, a blood lipid reduction activity in the cardiovascular system, and a blood circulation promoting activity.

A persimmon leaf extract according to the present invention means an ingredient extracted from persimmon leaves. The persimmon leaf extract has characteristics of allowing the skin to be elastic and strong and has an effect of protecting scalp and hair since the persimmon leaf extract is rich in vitamin A and vitamin C.

An aloe vera leaf extract according to the present invention means an ingredient extracted from aloe. The aloe vera leaf extract has effects of sterilization, lowering cholesterol level, stabilizing endocrine system, improving appetite, anti-cancer, detoxification, resistance to disease, anti-allergy, antihistamine, skin care, skin soothing, etc.

A chamomile flower extract according to the present invention, as an ingredient extracted from chamomile flower, may be referred to as a Matricaria flower extract. The chamomile flower may be used in bath, beauty care, fomentation, etc., and has effects in insect-proof, soothing, spasmolysis, pain relieving, diaphoresis, digestion promotion, fatigue recovery, etc.

A Portulaca oleracea L. extract according to the present invention means an ingredient extracted from Portulaca oleracea L. The Portulaca oleracea L. is also referred to as Portulaca, and means a landward annual weed which is frequently grown on the pavement. The Portulaca oleracea L. extract contains eicosapentaenoic acid, i.e., a type of omega-3 unsaturated fatty acid, and may provide a moisturizing effect and effects of improving skin resistance to external stimulation or bacteria and soothing the skin.

Argania spinosa kernel oil according to the present invention means oil extracted from fruits of Argania spinosa. The Argania spinosa kernel oil is excellent in a skin moisturizing effect, an antioxidant function, and an anti-inflammatory function by including omega 6, vitamin E, etc.

Niacinamide according to the present invention means a structure in which an amide group is bonded to niacin, and is a component of vitamin B3. The niacinamide has a whitening effect, and effects of improving skin tone, suppressing generation of troubles, and improving skin barrier.

Glyceryl acrylate/acrylic acid copolymer according to the present invention means a copolymer of glyceryl acrylate and acrylic acid. Although the glyceryl acrylate/acrylic acid copolymer may perform the role of a moisturizer preventing drying of the skin, the glyceryl acrylate/acrylic acid copolymer may be used even when adjusting viscosity of the soothing cream.

Hyaluronic acid according to the present invention includes amino acid and uronic acid and means a polymer compound including N-acetyl glucosamine and glucuronic acid. The hyaluronic acid may carry out synthesis of collagen within the skin, wrinkle removal, and antibacterial action.

Arginine according to the present invention, as a type of amino acid, may perform a role of storing moisture in the skin by being involved in activity of collagen.

Hydrolyzed collagen according to the present invention, as an aqueous potassium salt solution of a condensate of a collagen hydrolysate and coconut oil fatty acid, may be also used as a skin conditioner or a surfactant.

Sucrose distearate according to the present invention means an ingredient obtained by purifying raw sugar of sugar cane. Sucrose distearate of the soothing cream may be used as a surfactant and a skin conditioner.

Hydrogenated lecithin according to the present invention means an ingredient obtained by adding hydrogen to lecithin. The hydrogenated lecithin gives a help in skin care, and may also be used as a surfactant since the hydrogenated lecithin includes both a lipophilic group and a hydrophilic group.

Dimethicone according to the present invention, as a type of silicone oil, is not easily degraded compared to vegetable oil or mineral oil, has excellent usability, and may perform a role of holding moisture of the skin. The dimethicone may be used as a skin conditioner of the soothing cream, or a lubricant.

According to an embodiment of the present invention, although the skin conditioner may additionally include a material selected from the group consisting of butylene glycol, purified water, 1,2-hexanediol, glycerin, and combinations thereof, the present invention is not limited thereto.

Although butylene glycol, purified water, 1,2-hexanediol and glycerin included in the skin conditioner may be butylene glycol, purified water, 1,2-hexanediol and glycerin which are used together with the surfactant to dissolve active ingredients of the soothing cream, or which are for liquefying the active ingredients to supply the active ingredients to the skin, the present invention is not limited thereto.

According to an embodiment of the present invention, although the purified water may be purified water which is obtained by distilling water or passing the distilled water through an ion exchange resin, the present invention is not limited thereto.

According to an embodiment of the present invention, although the solvent may include purified water, glycerin, 1,2-hexanediol and butylene glycol, the present invention is not limited thereto.

According to an embodiment of the present invention, although the humectant may include a material selected from the group consisting of propylene glycol, glycerin and a combination thereof, the present invention is not limited thereto.

According to an embodiment of the present invention, although the soothing cream may further include a viscosity increasing agent including a material selected from the group consisting of carbomer, hydroxyethyl cellulose, ammonium acryloyldimethyltaurate/VP copolymer and combinations thereof, and a surfactant including PEG-60 hydrogenated castor oil and sucrose distearate, the present invention is not limited thereto.

Carbomer according to the present invention as an acidic polymer compound means a component in which acrylic acid is polymerized. The carbomer has advantages that it has excellent temperature stability and is less contaminated by microorganisms.

Hydroxyethyl cellulose according to the present invention means a modified cellulose polymer component having a hydroxyethyl branched-chain structure, and is excellent in salt resistance and pH stability.

Ammonium acryloyldimethyltaurate/VP copolymer according to the present invention, as a copolymer of ammonium acryloyldimethyltaurate and a vinyl pyrrolidone monomer, may be used as a water-soluble thickener, or forms a skin film and may give a soft touch to the skin.

PEG-60 hydrogenated castor oil according to the present invention means components of ethylene oxide and polyethylene glycol derivatives with an average 40 moles, and means a nonionic surfactant component derived from a product obtained by adding hydrogen to castor oil. The PEG-60 hydrogenated castor oil may also be used when giving a natural incense to the soothing cream.

According to an embodiment of the present invention, although the soothing cream may further include a preservative including chlorphenesin and a chelating agent including disodium EDTA, the present invention is not limited thereto.

Chlorphenesin according to the present invention, as an organic compound, is a type of chemical preservatives. The chlorphenesin may inhibit fungal breeding, and may effectively remove parasitic bacteria in the skin since antibacterial properties exist in the chlorphenesin.

Disodium EDTA according to the present invention means a component which prevents inter-ion bonding. The disodium EDTA may perform a role of giving a help such that a chelating agent for eluting trace amounts of metal ions within the soothing cream, a preservative, and the active ingredients are absorbed into the skin.

According to an embodiment of the present invention, although the soothing cream may further include menthol as the skin conditioner, the present invention is not limited thereto.

According to an embodiment of the present invention, although the soothing cream may include 0.1 to 2 parts by weight of the menthol based on 100 parts by weight of the soothing cream, the present invention is not limited thereto.

Menthol according to the present invention means an ingredient obtained by distilling leaves or stems of mint. The menthol helps circulation action of the skin and may give coolness and a feeling of refreshment to the skin.

The soothing cream relates to a soothing cream composition, without including the aforementioned materials, including: 5 to 20 wt % of a Maca extract; 3 to 15 wt % of glycerin; 3 to 15 wt % of butylene glycol; 0.1 to 3 wt % of lauryl glucoside; 0.1 to 3 wt % of betaine; 0.1 to 3 wt % of allantoin; 0.1 to 3 wt % of menthoxypropanediol; 0.01 to 0.5 wt % of a pH adjusting agent; 0.1 to 1 wt % of an antimicrobial agent; 0.1 to 1 wt % of a thickener; emulsifiers including 0.1 to 5 wt % of polyglyceryl-10 stearate, 0.1 to 5 wt % of polyglyceryl-3 methylglucose distearate and 0.1 to 5 wt % of polyglyceryl-3 diisostearate; oils including 0.1 to 5 wt % of isopropyl myristate, 0.1 to 5 wt % of Macadamia oil, 0.1 to 5 wt % of silicone oil and 0.1 to 5 wt % of perfluoropolyether; 0.5 to 10 wt % of a block copolymer represented by the following chemical formula 1; and a balance of water:

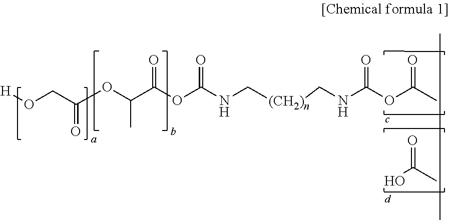

In chemical formula 1, n is a natural number of 2 to 10, and in a to d which show a mole fraction, a is 0.5, b is 0.5, c is 0.05 to 0.2, d is 0.8 to 0.95, and c+d is 1.

Although the soothing cream may include an antimicrobial agent including CS-BION, and a surfactant including a block copolymer of poly(D,L-lactide-co-glycolide) represented by the chemical formula 1 and polyacrylic acid substituted with a diisocyanate compound, the present invention is not limited thereto.

The block copolymer of poly(D,L-lactide-co-glycolide) and polyacrylic acid substituted with the diisocyanate compound may have a weight average molecular weight range of 10,000 to 100,000, more preferably 30,000 to 70,000. When the block copolymer satisfies the aforementioned weight average molecular weight range, the copolymer is favorable to the formation of a nanoemulsion.

It is preferable that the copolymer has an average particle diameter range of 10 to 100 nanometers. When the copolymer has such a particle size range, the copolymer may be excellent in penetration into the skin and collectability of active ingredients. The copolymer obtained as described above may be remarkably stable in an aqueous solution compared with low molecular weight micelles.

In the block copolymer of poly(D,L-lactide-co-glycolide) and polyacrylic acid substituted with the diisocyanate compound, poly(D,L-lactide-co-glycolide) (the following chemical formula 2) and polyacrylic acid substituted with the diisocyanate compound (the following chemical formula 3) may be formed in a bond of an isocyanate group and a hydroxy group of a carboxyl group:

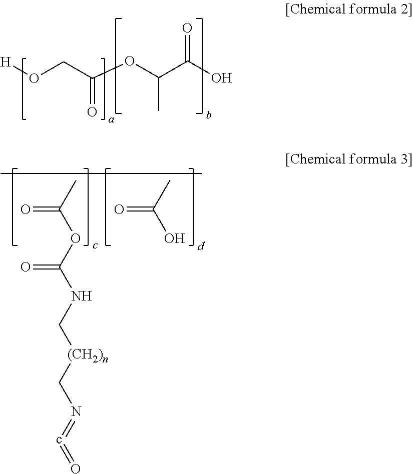

Further, the soothing cream may further include 3 to 10 wt % of a hot water extract of ingredients including 0.1 to 3 parts by weight of Mastic gum, 0.1 to 3 parts by weight of a Boswellia serrata powder, 0.1 to 3 parts by weight of an erythritol powder, 0.1 to 3 parts by weight of a Sophora tonkinensis Gapnep powder and 0.1 to 3 parts by weight of a Lobelia chinensis powder.

The Mastic (scientific name: Pistacia lentiscus LINNE) is a food additive registered as a natural flavoring substance No. 272 in Korean Food Additives Codex, and Mastic gum is a viscous natural gummi as a sap collected from Anacardiaceae mastic trees which are native to Chios island located in southeastern Greece. An ingredient of Mastic has been known to contain 60 to 70% of resin, 33% of αβ-Boswellic acid, 33% of olibanoresene, Arabic acid, etc.

A Mastic extract has been known to have an anti-inflammatory effect, antioxidant effect and anticancer effect, and has been extensively used in treatment or prevention of inflammatory diseases including arthritis, asthma and inflammatory bowel disease in Korean Medicine. History of Mastic has shown that Ancient Greeks had been known to use Mastic for the purpose of alleviating dysphoria in epigastrium, stomachalgia, dyspepsia and gastric ulcer or use Mastic as an ingredient of a sweetener or beverage, the Arabs have used Mastic oil as food, and Mastic has been known to be frequently used as a beverage or food ingredient even in Iraq. It has been mentioned in “Korean medicine clinical application” of Ahn Deokgyun that, if Mastic prepared in the form of a powder is applied to an affected area when an ulcer is caused by skin infection due to external injury, the Mastic promotes the formation of granulation tissue and removes ache, and is mainly used for the purpose of pain relieving.

The aforementioned Boswellia serrata is native to Africa, Southern Arabia, India, etc., and a green sap is flown out from the cut stem of the Boswellia serrata when cutting a stem of the Boswellia serrata. A Boswellia serrata extract has traditionally been used in inflammation such as a skin rash, an ulcer or the like, or a respiratory disease such as asthma, bronchitis or laryngitis, and has been known to have a medical effect even in arthritis, etc. The Boswellia serrata is a typical plant of Boswellia serrata genus, and the Boswellia serrata extract has been known to exhibit very excellent effects of inhibiting elastase activity and inhibiting decomposition of glycosaminoglycan by containing a large amount of Boswellic acid.

The aforementioned Sophora tonkinensis Gapnep is a rambling shrub root which is also called Menispermum dahuricum DC. The aforementioned Sophora tonkinensis Gapnep has been known to inhabit at sunny hillsides in all parts of Korea and contain alkaloid, dauricine, tetrandrine, and the like in roots and stems thereof (Yakugawa Zasshi 1970). Further, sinomenine and dauricine have been known to have antispasmodic activity and have efficacy even in hypertension and anti-inflammatory action, and an access to a rheumatism therapeutic agent is currently being researched by extracting and fractionating total alkaloids (Acta Med Okayama 1976).

The aforementioned Lobelia chinensis contains various bioactive substances as a medicinal plant used as an herb medicine in Korean Medicine. The aforementioned Lobelia chinensis is referred to as Lobelia chinensis Lour., Banbyeonran or geubhaesaeg, is distributed in south central region and Cheju Island of Korea, and is inhabited in a gully, a stream, or a paddy field marsh. The Lobelia chinensis powder has traditionally had efficacies of diuresis, anti-inflammation, detoxification and others, and has been known to be used as an herbal medicine for treating asthma, dyspnea, pertussis, malignant tumor, eczema and traumatic-hemorrhagic.

The soothing cream may further include any one or more selected from the group consisting of perfume, a coloring agent, a preservative, an oxidation stabilizer, etc. within a range that does not inhibit effects of the composition in addition to the aforementioned components.

Furthermore, a second aspect of the present invention provides a method of manufacturing a soothing cream, the method including the steps of: preparing a first mixture including purified water, propylene glycol, glycerin, a Maca root extract, butylene glycol, 1,2-hexanediol, niacinamide, a snail mucus filtrate, glyceryl acrylate/acrylic acid copolymer, hyaluronic acid, arginine, a Morus alba root extract, a green tea extract, a persimmon leaf extract, an aloe vera leaf extract, a chamomile flower extract, a Portulaca oleracea L. extract, hydrolyzed collagen, a viscosity increasing agent, a chelating agent, and a preservative; mixing a second mixture including ethanol, 1,2-hexanediol, PEG-60 hydrogenated castor oil, perfume and Argania spinosa kernel oil with the first mixture; and mixing a third mixture including hydrogenated lecithin, purified water, glycerin, dimethicone, and sucrose distearate with the first mixture.

With regard to a method of manufacturing a soothing cream according to a second aspect of the present invention, detailed descriptions have been omitted with respect to parts of the second aspect overlapped with those of the first aspect of the present invention. However, contents of descriptions described in the first aspect of the present invention may be equally applied to the second aspect of the present invention although the descriptions have been omitted in the second aspect.

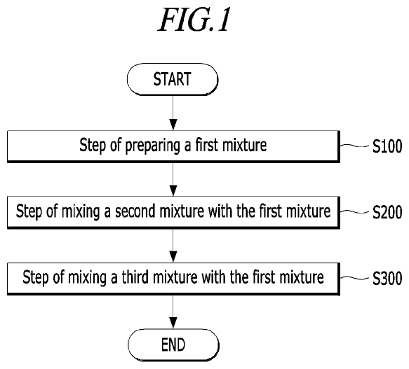

FIG. 1 is a flow chart showing a method of manufacturing a soothing cream according to an embodiment of the present invention.

First, the method includes a step (S100) of preparing a first mixture.

According to an embodiment of the present invention, although the step of preparing the first mixture may include the steps of: preparing a first material including the Maca root extract, butylene glycol, purified water and 1,2-hexanediol; preparing a second material including the glycerin, purified water, glyceryl acrylate/acrylic acid copolymer and 1,2-hexanediol; preparing a third material including the purified water, butylene glycol, Morus alba root extract, green tea extract, persimmon leaf extract and aloe vera leaf extract; and mixing the first material, second material, third material, purified water, propylene glycol, glycerin, niacinamide, snail mucus filtrate, hyaluronic acid, arginine, chamomile flower extract, Portulaca oleracea L. extract, hydrolyzed collagen, viscosity increasing agent, chelating agent and preservative, the present invention is not limited thereto.

According to an embodiment of the present invention, although the step of preparing the first mixture may be performed at a temperature of 65 to 80° C., the present invention is not limited thereto.

Subsequently, the method includes a step (S200) of mixing the second mixture with the first mixture.

According to an embodiment of the present invention, although the step of mixing the second mixture with the first mixture may further include a step of preparing the second mixture, the present invention is not limited thereto.

According to an embodiment of the present invention, although the step of preparing the second mixture may be performed at a temperature of 40 to 50° C., the present invention is not limited thereto.

According to an embodiment of the present invention, although the step of mixing the second mixture with the first mixture may include performing a stirring process at 1,500 rpm for 5 minutes, the present invention is not limited thereto.

Subsequently, the method includes a step (S300) of mixing the third mixture with the first mixture.

With regard to this, the first mixture to be mixed with the third mixture means that components of the second mixture are included in the first mixture.

According to an embodiment of the present invention, although the step of mixing the third mixture with the first mixture may further include a step of preparing the third mixture, the present invention is not limited thereto.

According to an embodiment of the present invention, although the step of preparing the third mixture may be performed at a temperature of 40 to 50° C., the present invention is not limited thereto.

According to an embodiment of the present invention, although the step of mixing the third mixture with the first mixture may include performing a stirring process at 1,500 rpm for 3 minutes, the present invention is not limited thereto.

According to an embodiment of the present invention, although the method may include the steps of mixing the first mixture, the second mixture and the third mixture, cooling a resulting mixture at 30° C., and conducting a QC test, the present invention is not limited thereto.

Meanwhile, the method of manufacturing the soothing cream, without including the aforementioned steps of the method, may include:

a step (a) of mixing an emulsifier including 0.1 to 5 wt % of polyglyceryl-10 stearate, 0.1 to 5 wt % of polyglyceryl-3 methylglucose distearate and 0.1 to 5 wt % of polyglyceryl-3 diisostearate with oil including 0.1 to 5 wt % of isopropyl myristate, 0.1 to 5 wt % of macadamia oil, 0.1 to 5 wt % of silicone oil and 0.1 to 5 wt % of perfluoropolyether with respect to the total weight of a composition to obtain a mixture, and heating the mixture to a temperature of 75 to 85° C. to prepare an oil phase part;

a step (b) of mixing 5 to 20 wt % of a Maca extract, 1 to 5 wt % of lauryl glucoside, 0.1 to 3 wt % of menthoxypropanediol, 0.1 to 1 wt % of an antimicrobial agent, 0.5 to 10 wt % of a block copolymer represented by the following chemical formula 1, and 30 to 70 wt % of water with respect to the total weight of the composition to obtain a mixture, and heating the mixture to a temperature of 75 to 85° C. to prepare a water phase part;

a step (c) of stirring the water phase part to 2,500 to 3,500 rpm within an emulsification tank, and injecting the oil phase part into the emulsification tank to stir the oil phase part to 2,500 to 3,500 rpm, thereby emulsifying the stirred materials to form a micro-sized first emulsion phase;

a cooling step (d) of cooling the first emulsion phase to a temperature of 37 to 50° C.;

a step (e) of injecting the first emulsion phase into a high pressure type emulsification machine at a temperature of 37 to 50° C. and secondly emulsifying the first emulsion phase to a pressure of 600 to 1,500 bars two to three times to obtain a secondary emulsion;

a step (f) of mixing 3 to 15 wt % of glycerin, 3 to 15 wt % of butylene glycol and 0.1 to 1 wt % of a thickener with the secondary emulsion of the step (e) to obtain a mixture; and

a step (g) of injecting 0.01 to 0.5 wt % of a pH adjusting agent into the mixture to adjust a pH value of the mixture to 6 to 7.

The method may include the step of additionally adding 3 to 10 wt % of a hot water extract of ingredients including 0.1 to 3 parts by weight of Mastic gum, 0.1 to 3 parts by weight of a Boswellia serrata powder, 0.1 to 3 parts by weight of an erythritol powder, 0.1 to 3 parts by weight of a Sophora tonkinensis Gapnep powder and 0.1 to 3 parts by weight of a Lobelia chinensis powder to the water phase part of the step (b).

Hereinafter, the present invention will be described in more detail through Examples, but the Examples are only for the purpose of describing the present invention, and the scope of the present invention is not limited thereto.

### Example 1

A first material including a Maca root extract, butylene glycol, purified water and 1,2-hexanediol was prepared, a second material including glycerin, purified water, glyceryl acrylate/acrylic acid copolymer and 1,2-hexanediol was prepared, and a third material including purified water, butylene glycol, a Morus alba root extract, a green tea extract, a persimmon leaf extract and an aloe vera leaf extract was prepared. Subsequently, a first mixture was prepared by increasing temperature of the mixture to 70 to 75° C. after mixing the first material to the third material, purified water, propylene glycol, glycerin, niacinamide, a snail mucus filtrate, hyaluronic acid, arginine, a chamomile flower extract, a Portulaca oleracea L. extract, hydrolyzed collagen, a viscosity increasing agent, a chelating agent, and a preservative to obtain a mixture.

A second mixture was prepared by uniformly mixing ethanol, 1,2-hexanediol, menthol, and PEG-60 hydrogenated castor oil, perfume, and Argania spinosa kernel oil at a temperature of 40 to 45° C. Subsequently, after injecting the second mixture into the first mixture, the first and second mixtures were stirred to 1,500 rpm for 5 minutes to obtain a material having the first and second mixtures mixed therein.

After uniformly mixing hydrogenated lecithin, purified water, glycerin, dimethicone, and sucrose distearate at a temperature of 40 to 45° C. to obtain a mixture, and injecting the mixture into the material having the first and second mixtures mixed therein to obtain a resulting material, the resulting material was stirred to 1,500 rpm for 3 minutes and cooled to 30° C.

### Example 2

After putting 1 kg of a Maca powder and 4 L of water into a heating container, boiling the Maca powder and water to 100° C. for 2 hours, and filtering the Maca powder through a filter paper to obtain a Maca powder-filtered solution, a Maca extract was prepared by boiling the Maca powder-filtered solution until 1 L of an extracted solution was obtained and concentrating the extracted solution.

Subsequently, after putting 300 g of Mastic gum, 300 g of a Boswellia serrata powder, 200 g of an erythritol powder, 100 g of a Sophora tonkinensis Gapnep powder, 100 g of a Lobelia chinensis powder, and 4 L of water into the heating container, boiling the Mastic gum, powders and water to 100° C. for 2 hours, and filtering the powders through a filter paper to obtain a powder-filtered solution, a mixed ingredient extract was prepared by boiling the powder-filtered solution until 1 L of an extracted solution was obtained and concentrating the extracted solution.

In order to produce polyacrylic acid substituted with a diisocyanate compound, 40 parts by weight of acrylic acid were injected into a 1 L reactor through which nitrogen gas was refluxed, and which had a cooling device installed therein to facilitate temperature control. After injecting 60 parts by weight of ethyl acetate (EAc) as a solvent into the reactor, a purging process was performed by using the nitrogen gas for 30 minutes. After maintaining the temperature of the reactor to 60° C. and injecting 0.1 part by weight of azobisisobutyronitrile (AIBN), i.e., a reaction initiator into the reactor, an acrylic acid polymer with a weight average molecular weight of 1,500 was manufactured by reacting the acrylic acid, EAc and AIBN for 3 hours.

After injecting 100 parts by weight of ethyl acetate (EAc) as a solvent into a 1 L reactor through which nitrogen gas was refluxed, and which had a cooling device installed therein to facilitate temperature control, 100 parts by weight of a solid of the above-manufactured acrylic acid polymer, 10 parts by weight of hexamethylene diisocyanate, 54 mg of magnesium trifluorosulfonate, 0.5 part by weight of Ionol, and 0.15 part by weight of Tinuvin were injected into the reactor. The materials injected into the reactor were heated to 110° C. by performing a heating process. Acrylic acid substituted with hexamethylene diisocyanate was manufactured by performing the heating process for 15 hours.

In order to manufacture a block copolymer of poly(D,L-lactide-co-glycolide) and polyacrylic acid substituted with a diisocyanate compound, after injecting 100 ml of dimethylformamide and 290 mg of N,N-carbonyldiimidazole (CDI) into a reaction container, dimethylformamide and CDI were stirred in the reaction container. A solution was prepared by adding 20 g of poly(D,L-lactide-co-glycolide) with a number average molecular weight of 11,000 (RG502, Boehringer Ingelheim, Germany) to a mixture of dimethylformamide and CDI, thereby activating a hydroxy end group of poly(D,L-lactide-co-glycolide). After adding 0.5 mg of Ionol, 0.15 mg of Tinuvin and 25 g of the above-manufactured polyacrylic acid with a number average molecular weight of 2,000 to the solution, the Ionol, Tinuvin and polyacrylic acid added to the solution were heated to 110° C. After performing a heating process for 15 hours, filtering the heated material with a nylon filter with a pore size of 0.45 micrometer, and dispersing the filtered material in deionized water, a block copolymer with a weight average molecular weight of 54,000 was manufactured by removing unreacted materials and a reaction reagent through dialysis.

In order to prepare an oil-in-water type nanoemulsion soothing cream composition, after mixing an emulsifier including 3 wt % of polyglyceryl-10 stearate, 3 wt % of polyglyceryl-3 methylglucose distearate and 3 wt % of polyglyceryl-3 diisostearate with oil including 3 wt % of isopropyl myristate, 3 wt % of macadamia oil, 3 wt % of silicone oil and 3 wt % of perfluoropolyether with respect to the total weight of the composition to obtain a mixture, and the mixture was heated to a temperature of 75 to 85° C. to prepare an oil phase part.

After mixing 10 wt % of a Maca extract, 2 wt % of lauryl glucoside, 2 wt % of menthoxypropanediol, 0.5 wt % of CS-BION as an antimicrobial agent, 5 wt % of the block copolymer represented by chemical formula 1 manufactured in the aforementioned manufacturing example 3, and 49.7 wt % of water with respect to the total weight of the composition to obtain a mixture, the mixture was heated to a temperature of 75 to 85° C. to prepare a water phase part.

After stirring the water phase part to 2,500 to 3,500 rpm within an emulsification tank, injecting the oil phase part into the emulsification tank to stir the oil phase part to 2,500 to 3,500 rpm, and emulsifying the stirred materials, a micro-sized first emulsion phase was formed. The first emulsion phase was cooled to a temperature of 37 to 50° C. After injecting the first emulsion phase into a high pressure type emulsification machine at a temperature of 37 to 50° C., the first emulsion phase was secondly emulsified to a pressure of 600 to 1,500 bars two to three times to obtain a secondary emulsion.

After mixing the secondary emulsion with 5 wt % of glycerin, 4 wt % of butylene glycol, and 0.5 wt % of Carbomer 980 as a thickener to obtain a mixture, a soothing cream composition was prepared by injecting 0.3 wt % of citric acid as a pH adjusting agent into the mixture, thereby adjusting a pH value of a resulting material to 6 to 7.

### Example 3

A soothing cream composition was prepared by the same processes as in Example 2 except that 5 wt % of the mixed ingredient extract was additionally added, and 44.7 wt % of water was used.

### Experimental Example 1

An experiment was conducted by allowing 200 adult men aged 25 to 55 to apply a soothing cream according to the present invention to their sexual organs. The experiment was progressed for total 7 weeks, and results were obtained by conducting interviews with experimentees on a weekly basis during the experiment period.

TABLE 1

| | First | Second | Third |
|---|---|---|---|
| Week | question | question | question |
| First week | Δ | Δ | Δ |
| Second week | ∘ | ∘ | x |
| Third week | ∘ | ∘ | Δ |
| Fourth week | ∘ | ∘ | ∘ |
| Fifth week | ∘ | ∘ | ∘ |
| Sixth week | ∘ | ∘ | ∘ |
| Seventh week | ∘ | ∘ | ∘ |

With regard to this, the first question means if there is a change in length of his sexual organ by his own feeling, the second question means if he himself can visually confirm a change in length of his sexual organ, and the third question means if others can also feel a change in length of his sexual organ. Further, with regard to the first to third questions, ∘ means exist, Δ means uncertain, and x means none.

Results of Table 1 were drawn up based on contents responded by 70% or more of the subjects. Referring to Table 1, when applying a soothing cream according to the present invention to men's sexual organs, it can be confirmed that lengths of the sexual organs are extended after 4 weeks.

### Experimental Example 2

Particle sizes of an emulsion of the soothing cream composition according to Example 2 were measured, and nano-emulsion stability was evaluated by a change in the particle sizes.

After respectively measuring particle sizes of water-in-oil type nanoparticle emulsion cosmetic compositions according to the present invention obtained in Example 2 by Mastersizer 2000 (Malvern Instrument, UK) using dynamic light scattering principle, measurement results are shown in the following Table 2. At this time, a process of measuring the particle sizes was performed under the following conditions after diluting the water-in-oil type nanoparticle emulsion cosmetic compositions with purified water such that turbidity values of nanoemulsion-diluted solutions became 10 to 20%.

Measurement time: 2 minutes, the number of measurements per second: 5×10³, temperature: 20° C., viscosity: 0.89 centipoise, particle refractive index: 1.4, dispersion medium refractive index: 1.33

After performing a process of measuring particle sizes twice, i.e., one day after preparation of the compositions and after storing the compositions at 40° C. for 6 months, and evaluating thermodynamic stability values of nanoemulsions after storing the compositions at high temperatures for a long period of time, measurement and evaluation results are shown in the following Table 2.

### [Experimental Example 3]: Emulsification Stability Observed by the Naked Eye

After performing a process of evaluating the emulsification stability values by visually observing emulsification stability values of the compositions as follows along with a process of measuring particles sizes of the water-in-oil type nanoparticle emulsion cosmetic compositions obtained in Example 2, measurement and evaluation results are shown in the following Table 2.

Emulsification states of the compositions of Example 2 at room temperature (about 25° C.) right after the preparation and those after storage at 40° C. for 6 months were comparatively observed. Unstable states including portions having sedimentation, separation, drainage, creaming, union or the like occurred therein were observed with the naked eye. Emusification stability is evaluated by percentage (%) of a stable portion except for an unstable portion from the whole portion, and is shown in the following equation.

Emulsification stability (%)=[(the whole portion−unstable portion)/the whole portion]×100%

TABLE 2

| | Particle size (nm) | | | Emulsification |
|---|---|---|---|---|
| Nanoparticle | After storing at | After storing at | | stability |
| emulsion | room temperature | 40° C. for 6 | Increase | observed by |
| cosmetic | for one day since | months since | in particle | the naked |
| composition | preparation | preparation | sizes (%) | eye (%) |
| Example 2 | 101 | 105 | 4.0 | 94 |

### [Experimental Example 4]: Effect of Inhibiting Secretion of IL-1α from HaCaT Keratinocyte

A biosynthesis amount of IL-1α causing erythema generated in the skin was confirmed by performing an SDS treatment process. Namely, the production amounts of IL-1α were expressed as a percentage after obtaining production amounts of IL-1α of respective samples when the amount of IL-1α when skin cells were treated with SDS only was defined as 100.

After inoculating HaCaT keratinocyte (German Cancer Research Institute, Germany) in a 24-well plate using DMEM (Dulbecco's Modified Eagle's Medium, Gibco) to which 10% FBS had been added to a density of 2×105 cells/well, the HaCaT keratinocyte inoculated in the 24-well plate was cultured in a humidified culture medium under conditions of 37° C. and 5% CO₂ for 1 day to obtain a first cultured solution. After exchanging the DMEM with serum-free DMEM, treating the first cultured solution with the Maca extract, the mixed ingredient extract, and a mixture of the Maca extract and the mixed ingredient extract (weight ratio of 1:1) in an amount of 100 ppm to obtain an extract-treated solution, culturing the extract-treated solution for 1 day to obtain a secondly cultured solution, and treating the secondly cultured solution with SDS (sodium dodecyl sulfate, Sigma) to obtain a SDS-treated solution, the SDS-treated solution was additionally cultured for 4 hours to obtain a third cultured solution.

After 4 hours, a portion of the third cultured solution was taken and used in quantification of IL-1α and protein. IL-1α quantification was performed according to an IL-1α quantification method of the kit by using a human IL-1α kit manufactured by Endogen. After quantifying protein by the BCA method of Sigma Corporation, thereby quantifying IL-1α with the kit, an IL-1α secretion amount per unit protein was measured by correcting the IL-1α quantified with the kit using a protein amount. Ketopropen which had conventionally been used as a skin irrigation-alleviating material was used as a positive control group, and a final IL-1α production rate (%) was obtained by the following mathematical equation. Results are shown in the following Table 2.

IL-1α production rate (%)=(A/B)×100

A: IL-1α production amount of sample

B: IL-1α production amount of sample treated with SDS only

TABLE 3

| | IL-1α |
|---|---|
| | production |
| Sample name | rate (%) |
| Control group | 34.12 ± 2.14 |
| Group treated with SDS only | 100.00 |
| Maca extract | 87.53 ± 3.52 |
| Mixed ingredient extract | 89.26 ± 1.12 |
| Mixture of the Maca extract and the mixed | 79.24 ± 1.31 |
| ingredient extract (weight ratio of 1:1) | |

Referring to the aforementioned Table 3, it can be seen that IL-1α production amount is remarkably reduced in samples of the Maca extract, the mixed ingredient extract and the mixture of the Maca extract and the mixed ingredient extract (weight ratio of 1:1) prepared in Example 2. This shows that samples of the present invention exhibit excellent effects of relieving troubles of erythema or the like which may be generated in the skin, and specifically exhibit excellent effects of soothing the skin and relieving skin irritation. Particularly, it can be seen that a synergistic effect is generated since the mixture sample exhibits an appreciably excellent effect compared to the Maca extract or the mixed ingredient extract.

### [Experimental Example 5]: Effect of Promoting Collagen Synthesis

CCD-986sk cells, i.e., human fibroblastoma were used in a DMEM culture medium to which 10% fetal bovine serum (FBS, Gibco Co.), 100 μg/ml of streptomycin, and 100 U/ml of penicillin had been added under conditions of 37° C. and 5% CO₂. After sufficiently proliferating the CCD-986sk cells in a 75 cm² flask (Falcon, USA), washing the surface of cultured cells with a phosphate buffer saline (PBS) solution at intervals of 2 to 3 days of culturing, and injecting a 0.25% trypsin-EDTA solution into the cultured cells washed with the PBS solution, the cultured cells having the 0.25% trypsin-EDTA solution injected thereinto were treated in an incubator for 1 to 2 minutes to desorb the cells. The desorbed cells were cultured to a split ratio of 1:20 under conditions of 37° C. and 5% CO₂ by moving 15 ml of the DMEM culture medium containing the 10% FBS to a new culture container.

After dividing the CCD-986sk cells to a density of 5×104 cells/well on a 24-well plate to check a collagen synthesis effect, culturing the divided CCD-986sk cells with a 10% FBS/SMEM culture medium for 24 hours, and adding a gradually diluted sample to a serum free culture medium, the gradually diluted sample added to the serum free culture medium was again cultured in a CO₂ incubator for 24 hours to obtain a cell culture solution. An amount of collagen was measured from the cell culture solution by using Procollagen Type C-peptide EIA kit (Takara, Japan). First, after adding 100 μl of an antibody-POD conjugate solution to each of wells, injecting each of the Maca extract, the mixed ingredient extract, and a mixture of the Maca extract and the mixed ingredient extract (weight ratio of 1:1) as a sample as much as 20 μl into each of the wells, mixing the sample well with the antibody-POD conjugate solution to perform a reaction process at 37° C. for 3 hours, and removing contents from a reaction product when the reaction process was completed, a resulting sample material was washed with 400 μl of PBS four times. After removing remaining PBS from the resulting sample material, injecting 100 μl of a matrix solution into each of the wells, reacting the sample material with the matrix solution at 20° to 30° C. for 15 minutes, and adding 100 μl of 1 M H₂SO₄ to a reaction product to stop color development of the reaction product, the color development-stopped reaction product was shaken to obtain a resulting material. After measuring an absorbance value of the resulting material at 450 nm, a collagen amount was calculated by drawing a standard concentration curve. After calculating rates of collagen biosynthesis by the following equation, the calculation results are shown in the following Table 4.

RCB (%)=(A/B)×100

A: collagen cpm

B: (Total collagen cpm−collagen cpm)×5.4+collagen cpm

TABLE 4

| | Rate of collagen |
|---|---|
| | biosynthesis |
| Sample name | (RCB) (%) |
| Non-treated control group | 100 |
| Maca extract | 127 |
| Mixed ingredient extract | 122 |
| Mixture of the Maca extract and the mixed | 143 |
| ingredient extract (weight ratio of 1:1) | |

As shown in the aforementioned Table 4, it can be confirmed that collagen synthesis amounts are remarkably increased in samples of the Maca extract, the mixed ingredient extract, and the mixture of the Maca extract and the mixed ingredient extract (weight ratio of 1:1). Particularly, it can be seen that a synergistic effect is generated since the sample of the mixture of the Maca extract and the mixed ingredient extract (weight ratio of 1:1) exhibits an appreciably excellent effect of increasing the collagen synthesis amount compared to the Maca extract or the mixed ingredient extract.

### [Experimental Example 6]: User Evaluation of Soothing Cream Compositions

Performance values for the soothing cream compositions of Examples 2 and 3 and performance value for the soothing cream (product name: Crema Caracol, manufacturer: Jaminkyung) of Comparative Example 1 were evaluated. The experiment was conducted in such a manner that the women were allowed to give 1 to 10 points with respect to usability, moisture sense, skin elasticity improvement, and wrinkle amelioration after selecting 30 women in their 30s as experience groups, dividing 30 women into 3 groups of 10 people per group of the women, thereby allowing each 3 groups of 10 people per group to use the soothing cream compositions of Example 2, Example 3 and Comparative Example 1 twice (morning and evening) a day for 1 month.

After averaging the performance values given by each of the experience groups to obtain average performance value, the average performance value is shown in the following Table 5.

TABLE 5

| | Comparative | | |
|---|---|---|---|
| | Example 1 | Example 2 | Example 3 |
| Usability | 8.5 | 9.1 | 9.2 |
| Moisture sense | 8.1 | 8.8 | 8.9 |
| Skin elasticity | 3.3 | 6.5 | 9.1 |
| improvement | | | |
| Wrinkle | 2.9 | 6.1 | 8.1 |
| amelioration | | | |
| Total | 5.7 | 7.6 | 8.8 |

Skin troubles were not generated in all of 30 experimentees while the aforementioned test was being conducted, all of the experimentees were participated in the test for 1 month. As can be confirmed from the aforementioned Table 5, the soothing creams in Examples 2 and 3 of the present invention exhibit remarkable effects even in moisture sense, skin elasticity improvement, and wrinkle amelioration as well as usability compared to the soothing cream in Comparative Example 1 on the market. Particularly, it is confirmed that the soothing cream in Example 3 provides a more excellent effect than that of Example 1 by exhibiting a synergistic effect.

Although the exemplary embodiments of the present invention have been described in detail with reference to the accompanying drawing, the present invention is not limited thereto and may be embodied in many different forms without departing from the technical concept of the present invention. Therefore, the exemplary embodiments of the present invention are provided for illustrative purposes only but not intended to limit the technical spirit of the present invention. The scope of the technical spirit of the present invention is not limited thereto. Therefore, it should be understood that the above-described exemplary embodiments are illustrative in all aspects and do not limit the present invention.

The protective scope of the present invention should be construed based on the following claims, and all the technical concepts in the equivalent scope thereof should be construed as falling within the scope of the present invention.

What is claimed are:

1. A method of treating a penis of male human using a composition including a Maca root extract as an active ingredient to enhance erection of the penis, the method comprising:
applying an effective amount of the composition to a skin of a penis of a male human in need thereof,
wherein the composition comprises: a skin conditioner, a solvent, a humectant, a viscosity increasing agent, a surfactant, a preservative, a chelating agent, and perfume,
wherein the skin conditioner includes: a Maca root extract, a natural extract, niacinamide, glyceryl acrylate/acrylic acid copolymer, hyaluronic acid, arginine, hydrolyzed collagen, sucrose distearate, hydrogenated lecithin, and dimethicone,
wherein the composition includes: 0.5 to 10 parts by weight of the Maca root extract; and 1 to 5 parts by weight of the natural extract based on 100 parts by weight of the composition,
wherein the Maca root extract stimulates artery positioned in the penis to promote blood circulation, and
wherein the natural extract includes: a material selected from the group consisting of a snail mucus filtrate, a Morus alba root extract, a green tea extract, a persimmon leaf extract, an aloe vera leaf extract, a chamomile flower extract, a Portulaca oleracea L. extract, Argania spinosa kernel oil, and combinations thereof.

2. The method of claim 1, wherein the skin conditioner further includes: a material selected from the group consisting of butylene glycol, purified water, 1,2-hexanediol, glycerin, and combinations thereof.

3. The method of claim 1, wherein the Maca root extract includes: a material selected from the group consisting of a Maca root powder, a Maca root hot water extract, and a combination thereof.

4. The method of claim 1, wherein the solvent includes: purified water, glycerin, 1,2-hexanediol and butylene glycol.

5. The method of claim 1, wherein the humectant includes: a material selected from the group consisting of propylene glycol, glycerin, and a combination thereof.

6. The method of claim 1, wherein the viscosity increasing agent includes: a material selected from the group consisting of carbomer, hydroxyethyl cellulose, ammonium acryloyldimethyltaurate/VP copolymer and combinations thereof, and a surfactant including PEG-60 hydrogenated castor oil and sucrose distearate.

7. The method of claim 1, wherein the preservative includes: chlorphenesin, and the chelating agent includes: disodium EDTA.

8. The method of claim 1, wherein the skin conditioner further includes: menthol.

9. The method of claim 8, wherein the menthol is included in 0.1 to 2 parts by weight based on 100 parts by weight of the composition.

10. A method of using a composition including a Maca root extract as an active ingredient to treat a sexual organ of male human, the method comprising:
applying an effective amount of the composition to a skin of a penis of a male human in need thereof,
wherein the composition comprises: a skin conditioner, a solvent, a humectant, a viscosity increasing agent, a surfactant, a preservative, a chelating agent, and perfume,
wherein the skin conditioner includes a Maca root extract, a natural extract, niacinamide, glyceryl acrylate/acrylic acid copolymer, hyaluronic acid, arginine, hydrolyzed collagen, sucrose distearate, hydrogenated lecithin, and dimethicone,
wherein the natural extract includes a material selected from the group consisting of a snail mucus filtrate, a Morus alba root extract, a green tea extract, a persimmon leaf extract, an aloe vera leaf extract, a chamomile flower extract, a Portulaca oleracea L. extract, Argania spinosa kernel oil, and combinations thereof,
wherein the composition includes: 0.5 to 10 parts by weight of the Maca root extract; and 1 to 5 parts by weight of the natural extract based on 100 parts by weight of the composition, and
wherein the Maca root extract stimulates artery positioned in the penis to promote blood circulation and enhance erection of the penis.

11. The method of claim 10, wherein the skin conditioner further includes a material selected from the group consisting of butylene glycol, purified water, 1,2-hexanediol, glycerin, and combinations thereof.

12. The method of claim 10, wherein the Maca root extract includes a material selected from the group consisting of a Maca root powder, a Maca root hot water extract, and a combination thereof.

13. The method of claim 10, wherein the solvent includes purified water, glycerin, 1,2-hexanediol and butylene glycol.

14. The method of claim 10, wherein the humectant includes a material selected from the group consisting of propylene glycol, glycerin, and a combination thereof.

15. The method of claim 10, wherein the viscosity increasing agent includes: a material selected from the group consisting of carbomer, hydroxyethyl cellulose, ammonium acryloyldimethyltaurate/VP copolymer and combinations thereof, and a surfactant including PEG-60 hydrogenated castor oil and sucrose distearate.

16. The method of claim 10, wherein the preservative includes: chlorphenesin, and the chelating agent includes: disodium EDTA.

17. The method of claim 10, wherein the skin conditioner further includes: menthol.

18. The method of claim 17, wherein the menthol is included in 0.1 to 2 parts by weight based on 100 parts by weight of the composition.
